(19) United States
(12) Patent Application Publication
(10) Pub. No.: US 2022/0346980 A1
(43) Pub. Date: Nov. 3, 2022

(54) MECHANICAL PROSTHETIC LIMB

(71) Applicants: Dafei Zu, Chongqing (CN); Rui Liu, Chongqing (CN); Rui Huang, Chongqing (CN); Zixiang Wang, Chongqing (CN)

(72) Inventors: Dafei Zu, Chongqing (CN); Rui Liu, Chongqing (CN); Rui Huang, Chongqing (CN); Zixiang Wang, Chongqing (CN)

(21) Appl. No.: 17/409,832

(22) Filed: Aug. 24, 2021

(30) Foreign Application Priority Data
Apr. 28, 2021 (CN) 202110469404.5

(51) Int. Cl. A61F 2/70; A61F 2/66

(52) U.S. Cl. CPC A61F 2/70; A61F 2/66; A61F 2002/6836

(57) ABSTRACT

The present invention provides a mechanical prosthetic limb, which relates to the technical field of medical devices. The mechanical prosthetic limb includes a prosthetic limb shank, a fixed component for connecting the prosthetic limb shank with a human body, a power-driven first drive mechanism that is installed on the prosthetic limb shank, and a prosthetic limb sole that is connected to the bottom of the prosthetic limb shank; the fixed component is rotationally connected with the prosthetic limb shank through the first drive mechanism; and the prosthetic limb shank is rotationally connected with the prosthetic limb sole through the first drive mechanism. It can relieve the burdens of the disabled in the automatic advance way, thereby improving convenience.

### TECHNICAL FIELD

The present invention relates to the technical field of medical devices, in particular to a mechanical prosthetic limb.

### BACKGROUND

Various injuries arising from diseases, natural disasters, traffic accidents, and other cases have caused hundreds of amputees to the society, and especially in recent several decades, with the frequent occurrence of industrial and traffic accidents, and natural disasters, the number of amputees is getting more and more. According to the latest statistical report published by the China Federation of the Disabled in 2012, the total number of all kinds of the disabled in China reached 85 million, in which lower-limb amputees accounted for 71%. The only way for these patients to stand up again is to be fitted with prosthetic limbs or with the assistance of other appliances. However, the current prosthetic limb devices are connected with residual limbs under negative pressure to fulfill the objective of supporting walking, that is, the residual limbs are accommodated in prosthetic limb acceptance cavities, and the residual limbs come in full contact with openings of the acceptance cavities hermetically, achieving the suspension of the acceptance cavities under negative pressure in the acceptance cavities. When patients wear the prosthetic limbs to stand, walk, and perform other basic activities, the gravity of human bodies is transferred to the ground through the prosthetic limb acceptance cavities, and meanwhile, the counter-acting force of the ground is also transferred to the prosthetic limbs from the prosthetic limb acceptance cavities, thereby attaining the motion function for the residual limbs to control the standing and walking of the prosthetic limbs. However, the existing prosthetic limbs have the disadvantages that due to the direct fixing of most of prosthetic limbs with the residual limbs, the prosthetic limbs are extremely stiff in movement when moving, and need to be driven by users, making the disabled take a long time to make adaptation, and causing harm to the residual limbs after being used for a long time.

### SUMMARY

The present invention aims to provide a mechanical prosthetic limb, which can relieve the burdens of the disabled in the automatic advance way, thereby improving convenience.

The embodiments of the present invention are achieved as below:

The embodiments of the present application provide a mechanical prosthetic limb, which includes a prosthetic limb shank, a fixed component for connecting the prosthetic limb shank with a human body, a power-driven first drive mechanism that is installed on the prosthetic limb shank, and a prosthetic limb sole that is connected to the bottom of the prosthetic limb shank; the fixed component is rotationally connected with the prosthetic limb shank through the first drive mechanism; and the prosthetic limb shank is rotationally connected with the prosthetic limb sole through the first drive mechanism.

In some embodiments of the present invention, the first drive mechanism includes a motor, a first differential gear, a first drive shaft, a first bevel gear, a second bevel gear, a second drive shaft, a third drive shaft, a third bevel gear, a fourth bevel gear, a fourth drive shaft, a fifth bevel gear, and a sixth bevel gear, where an output shaft of the motor is connected with the sixth bevel gear; the sixth bevel gear is meshed with the fifth bevel gear; the fifth bevel gear is connected with an input shaft of the first differential gear; one output shaft of the first differential gear is connected with the second bevel gear; the first bevel gear is meshed with the second bevel gear; the fixed component is connected with the second bevel gear through the third drive shaft; the other output shaft of the first differential gear is connected with the third bevel gear through the third drive shaft; the third bevel gear is meshed with the fourth bevel gear; the fourth bevel gear is connected with the fourth drive shaft; the fourth drive shaft is connected with the prosthetic limb sole after running through the prosthetic limb shank; and the fourth drive shaft is rotationally connected with the prosthetic limb sole.

In some embodiments of the present invention, the mechanical prosthetic limb further includes a prosthetic limb thigh rotationally connected with the prosthetic limb shank and a second drive mechanism installed inside the prosthetic limb thigh, where the second drive mechanism includes a second differential gear, a flexible drive part, and a fifth drive shaft; an input end of the second differential gear is connected with the prosthetic limb thigh; one output end of the second differential gear is connected with one end of the flexible drive part; the other end of the flexible drive part is connected with the sixth bevel gear through the fifth drive shaft; and the other output end of the second differential gear is connected with the fixed component.

In some embodiments of the present invention, the mechanical prosthetic limb further includes a universal shaft, where the flexible drive part is connected with the fourth drive shaft through the universal shaft.

In some embodiments of the present invention, a waterproof gasket is arranged at a rotational joint of the fourth drive shaft and the prosthetic limb sole.

In some embodiments of the present invention, the fixed component includes a flexible fixed shell and a fastening rope, where the fastening rope penetrates in the flexible fixed shell; and the fastening rope is movably connected with the flexible fixed shell.

In some embodiments of the present invention, an air vent is formed in the flexible fixed shell.

In some embodiments of the present invention, leather layers are arranged outside the prosthetic limb sole, the prosthetic limb shank, and the prosthetic limb thigh.

In some embodiments of the present invention, an anti-skidding boss is arranged on a contact surface between the prosthetic limb sole and the ground.

In some embodiments of the present invention, a cross section of the anti-skidding boss is in a triangular shape.

Compared with the prior art, the embodiments of the present invention at least have the following advantages:

The mechanical prosthetic limb includes a prosthetic limb shank, a fixed component for connecting the prosthetic limb shank with a human body, a power-driven first drive mechanism that is installed on the prosthetic limb shank, and a prosthetic limb sole that is connected to the bottom of the prosthetic limb shank; the fixed component is rotationally connected with the prosthetic limb shank through the first drive mechanism; and the prosthetic limb shank is rotationally connected with the prosthetic limb sole through the first drive mechanism.

In view of the direct fixing of a prosthetic limb with a residual limb in the prior art, the prosthetic limb is extremely stiff in movement when moving, bringing inconvenience to the action of a user, and needs to be driven by the user, making the disabled take a long time to make adaptation, and causing harm to the residual limbs after being used for a long time. To solve the previously described problem, in the embodiments, the prosthetic limb is driven electrically by the first drive mechanism installed on the prosthetic limb shank, and the prosthetic limb shank is respectively and rotationally connected with the fixed component and the prosthetic limb sole, so that the prosthetic limb shank can be raised under the control of the first drive mechanism, and meanwhile, tiptoes of the prosthetic limb sole incline downwards under the control of the first drive mechanism; and when the prosthetic limb sole lays down, tiptoes touch down to the ground firstly, thereby preventing impact on the prosthetic limb arising from the direct touchdown of the center of the prosthetic limb sole, ensuring the residual limb isn't hurt during long-term use, and improving the safety and comfort. In addition, the power-driven drive mechanism applied in design makes the user walk conveniently and fast, with less efforts, thereby improving the practicability.

### BRIEF DESCRIPTION OF DRAWINGS

In order to describe the technical solutions of the embodiments of the present invention more clearly, the drawings required to be used in the embodiments will be simply introduced below. Obviously, the drawings described below are only some embodiments of the present invention, which can't be deemed as the limit to the scope. Other drawings may further be obtained by a person of ordinary skill in the art according to these drawings without creative labor.

FIG. 1 is a structural schematic diagram illustrating a mechanical prosthetic limb, according to the present invention;

FIG. 2 is a structural schematic diagram illustrating another mechanical prosthetic limb, according to the present invention;

FIG. 3 is a structural schematic diagram illustrating a steering mechanism, according to the present invention;

FIG. 4 is a structural schematic diagram illustrating a prosthetic limb shank and a prosthetic limb thigh, according to the present invention.

Figure number: 1. Fixed component; 11. Flexible fixed shell; 12. Fastening rope; 13. Bearing; 2. Prosthetic limb thigh; 3. Second drive mechanism; 31. Second differential gear; 32. Flexible connecting part; 33. Fifth drive shaft; 4. Prosthetic limb shank; 5. First drive mechanism; 51. First bevel gear; 511. Second bevel gear; 512. Second drive shaft; 52. First drive shaft; 53. First differential gear; 54. Third drive shaft; 55. Third bevel gear; 551. Fourth drive shaft; 552. Fourth bevel gear; 56. Sixth bevel gear; 57. Fifth bevel gear; 58. Motor; 6. Prosthetic limb sole; 61. Anti-skidding boss; 7. Seventh bevel gear; 71. Eighth bevel gear.

### DESCRIPTION OF EMBODIMENTS

To make the objectives, technical solutions, and advantages of the embodiments of the present invention more clearly, the following describes the technical solutions in the embodiments of the present invention clearly and completely with reference to the drawings in the embodiments of the present invention. Clearly, the described embodiments are merely some but not all of the embodiments of the present invention. Generally, components of the embodiments of the present invention described and illustrated in the drawings here can be arranged and designed in different configurations.

Therefore, the following detailed descriptions of the embodiments of the present invention provided in the drawings are not intended to limit the scope of the present invention for which the protection is claimed, but only indicate the selected embodiments of the present invention. All other embodiments obtained by a person of ordinary skill in the art based on the embodiments of the present invention shall fall within the protection scope of the present invention, without contributing creative labor.

It is worthwhile to note that a similar mark number and a letter represent similar terms in the following drawings. Therefore, once certain term is defined in a drawing, it is unnecessary to describe and explain this term further in the subsequent drawings.

In the descriptions of the embodiments of the present invention, it is worthwhile to note that “up”, “down”, “horizontal”, “interior”, “exterior” and other terms indicating directions or positional relationships are directions or positional relationships shown based on the drawings, or the direction or positional relationship of the customary placement of a product of the present invention when used is only used to describe the present invention conveniently and simplify the description, rather than indicating or implying that a device or an element that is signified must be constructed and operated in a specified direction. Therefore, it can't be interpreted as limit to the present invention. In addition, terms “first”, “second”, “third”, etc. are only used to distinguish the description, which can't be interpreted to indicate or imply relative importance.

In addition, “horizontal”, and other terms don't mean that a component is required to be absolutely horizontal, but can be tilted slightly. For example, “horizontal” merely indicates the component keeps more horizontal relatively in the direction, and doesn't mean that the structure must keep horizontal completely, but can be tilted slightly.

In the descriptions of the embodiments of the present invention, “a plurality of” represents at least 2.

In the descriptions of the embodiments of the present invention, it is also worthwhile to note that unless otherwise specified and limited clearly, terms “arranged”, “installed”, and “connected” shall be interpreted broadly, for example, fixedly connected, detachably connected, or integrally connected is available; either mechanically connected or electrically connected is available; and directly connection, indirectly connected through an intermediate medium, or communicated inside two elements is also available. A person of ordinary skill in the art can understand the specific meaning of the previously described terms in the present invention based on the specific case.

### Embodiment 1

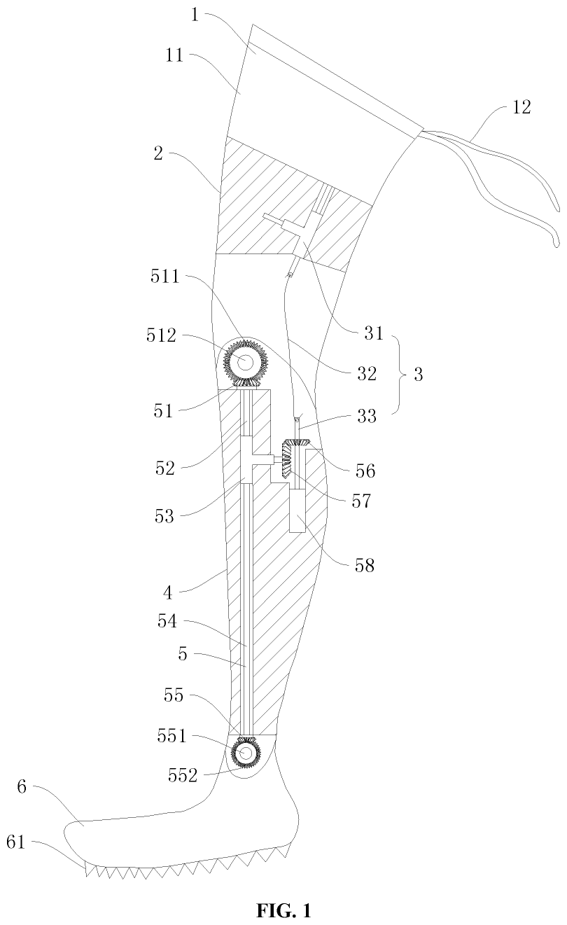

With reference to FIG. 1, the embodiment provides a mechanical prosthetic limb, which includes a prosthetic limb shank 4, a fixed component 1 for connecting the prosthetic limb shank 4 with a human body, a power-driven first drive mechanism 5 that is installed on the prosthetic limb shank 4, and a prosthetic limb sole 6 that is connected to the bottom of the prosthetic limb shank 4; the fixed component 1 is rotationally connected with the prosthetic limb shank 4 through the first drive mechanism 5; and the prosthetic limb shank 4 is rotationally connected with the prosthetic limb sole 6 through the first drive mechanism 5.

In some embodiments of the present invention, in view of the direct fixing of a prosthetic limb with a residual limb in the prior art, the prosthetic limb is extremely stiff in movement when moving, bringing inconvenience to the action of a user, and needs to be driven by the user, making the disabled take a long time to make adaptation, and causing harm to the residual limb after being used for a long time. To solve the previously described problem, in the embodiment, the prosthetic limb is driven electrically by the first drive mechanism 5 installed on the prosthetic limb shank 4, and the prosthetic limb shank 4 is respectively and rotationally connected with the fixed component 1 and the prosthetic limb sole 6, so that the prosthetic limb shank 4 can be raised under the control of the first drive mechanism 5, and meanwhile, tiptoes of the prosthetic limb sole 6 incline downwards under the control of the first drive mechanism 5; and when the prosthetic limb sole 6 lays down, tiptoes touches down to the ground firstly, thereby preventing impact on the prosthetic limb arising from the direct touchdown of the center of the prosthetic limb sole 6, ensuring the residual limb isn't hurt during long-term use, and improving the safety and comfort. In addition, the power-driven drive mechanism applied in design makes the user walk conveniently and fast, with less efforts, thereby improving the practicability.

### Embodiment 2

With reference to FIG. 1, based on the technical solution of the embodiment 1, the embodiment provides that a first drive mechanism includes a motor 58, a first differential gear 53, a first drive shaft 52, a first bevel gear 51, a second bevel gear 511, a second drive shaft 512, a third drive shaft 54, a third bevel gear 55, a fourth bevel gear 552, a fourth drive shaft 551, a fifth bevel gear 57, and a sixth bevel gear 56, where an output shaft of the motor 58 is connected with the sixth bevel gear 56; the sixth bevel gear 56 is meshed with the fifth bevel gear 57; the fifth bevel gear 57 is connected with an input shaft of the first differential gear 53; one output shaft of the first differential gear 53 is connected with the second bevel gear 511; the first bevel gear 51 is meshed with the second bevel gear 511; the fixed component 1 is connected with the second bevel gear 511 through the third drive shaft 54; the other output shaft of the first differential gear 53 is connected with the third bevel gear 55 through the third drive shaft 54; the third bevel gear 55 is meshed with the fourth bevel gear 552; the fourth bevel gear 552 is connected with the fourth drive shaft 551; the fourth drive shaft 551 is connected with the prosthetic limb sole 6 after running through the prosthetic limb shank 4; and the fourth drive shaft 551 is rotationally connected with the prosthetic limb sole 6.

In some embodiments of the present invention, the rotation direction between the fixed component 1 and the prosthetic limb is perpendicular to a user's face, since rotation between the fixed component 1 and the prosthetic limb sole 6 and the prosthetic limb shank 4 need to comply with ergonomics. Since a motor 58 is large in occupied dimension, the fifth bevel gear 57 and the sixth bevel gear 56 are arranged to make the motor 58 parallel to the third drive shaft 54, thereby avoiding the high bulging of the prosthetic limb shank 4 arising from large dimension in the motor 58, resulting in hindering to the daily use of the prosthetic limb. In addition, the objective of arranging the first differential gear 53 is that in terms of ergonomics, an rotational angle between the prosthetic limb sole 6 and the prosthetic limb shank 4 is different from that between the prosthetic limb shank 4 and the fixed component 1, that is, there is an angle difference between them, and thus, the first drive shaft 52 and the third drive shaft 54 rotate at different rotation speeds through the first differential gear 53 under the driving of the motor 58 to ensure the angle difference, thereby ensuring the prosthetic limb sole 6 and the prosthetic limb shank 4 can be in reasonably good fit with the walking of a human body in movement, and improving the comfort of a user. In addition, since the prosthetic limb needs to make reciprocating motion in order to achieve continuous advance, a servo motor 58 or a stepper motor 58 is applied, and a relay switch or an electromyography transducer is used to achieve the automatic control of the motor 58, thereby improving practicability further.

### Embodiment 3

With reference to FIG. 1, the embodiment, based on the technical solution of the embodiment 2, provides that the mechanical prosthetic limb further includes a prosthetic limb thigh 2 rotationally connected with a prosthetic limb shank 4 and a second drive mechanism 3 installed inside the prosthetic limb thigh 2, where the second drive mechanism 3 includes a second differential gear 31, a flexible drive part, and a fifth drive shaft 33; an input end of the second differential gear 31 is connected with the prosthetic limb thigh 2; one output end of the second differential gear 31 is connected with one end of the flexible drive part; the other end of the flexible drive part is connected with a sixth bevel gear 56 through the fifth drive shaft 33; and the other output end of the second differential gear 31 is connected with a fixed component 1.

In some embodiments of the present invention, for different disabled persons, the degree of leg amputation is not exactly the same. In order to increase the adaptive range of this design, the prosthetic limb thigh 2, the fifth drive shaft 33, and the second drive mechanism 3 installed inside the prosthetic limb thigh 2 are arranged to be suitable for the disabled with thigh amputation. In terms of the description of embodiments, the flexible drive part is connected with the sixth bevel gear 56 through the fifth drive shaft 33, so that the power of a motor 58 is transferred to one output shaft of the second differential gear 31 through the flexible drive part, and then, is outputted from the other output shaft of the second differential gear 31 for driving a fixed component 1 to fulfill the objective of making a thigh incline at a certain angle, thereby increasing the movable angles of the mechanical prosthetic limb, and improving flexibility, and practicability. In order to facilitate the flexible drive part for flexibility and supporting capacity during driving, an iron wire is used.

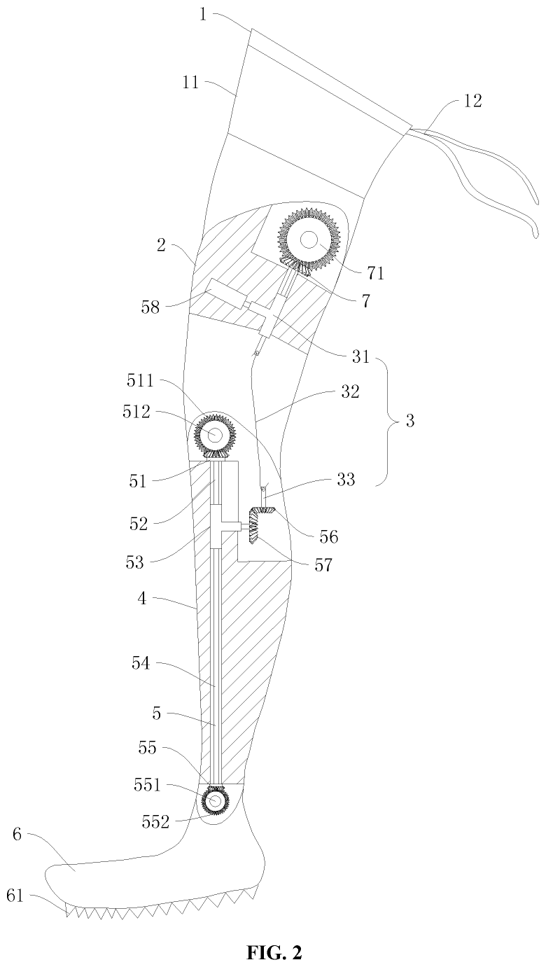

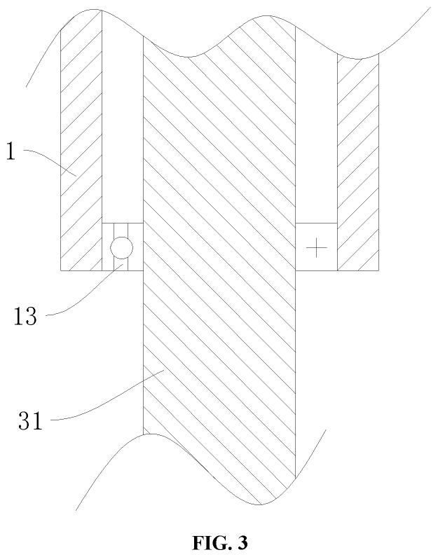

In some embodiments of the present invention, there is another implementation, as shown in FIG. 2, that the motor 58 is arranged in the thigh, so that a device is integrally composed of one motor 58, two differential gears, three drive joints (a hip joint, a knee joint, and an ankle joint), a flexible shaft that can transfer power, and a steel disc-shaped drive shaft that may deform, where a first differential gear 53 divides power into two sections, power is transferred to the hip joint (i.e., a seventh bevel gear 7 and an eighth bevel gear 71) at one end of the device, while power is transferred to the first differential gear 53 of the prosthetic limb shank 4 through a flexible connecting part 32 to drive the knee joint (i.e., the drive of a first gear and a second gear) and the ankle joint (i.e., the drive of a third gear and a fourth gear) to make motion; and a third drive shaft 54 that drives the ankle joint can be connected with an elastic strip-type steel disc, which can simulate the damping effect during walking, and reduce damage to a mechanical structure during high-strength operation. Meanwhile, a steering mechanism is arranged, as shown in the FIG. 3, which is achieved by a large bearing 13. In terms of the implementation, the prosthetic limb thigh 2 and the prosthetic limb shank 4 are respectively fixed on an inner ring and an outer ring of the bearing 13 to achieve steering on the ground like a human foot, without arranging a power device additionally. In addition, each joint is provided with a brake locking device, so that it can be controlled separately, while the used power effect keeps unchanged.

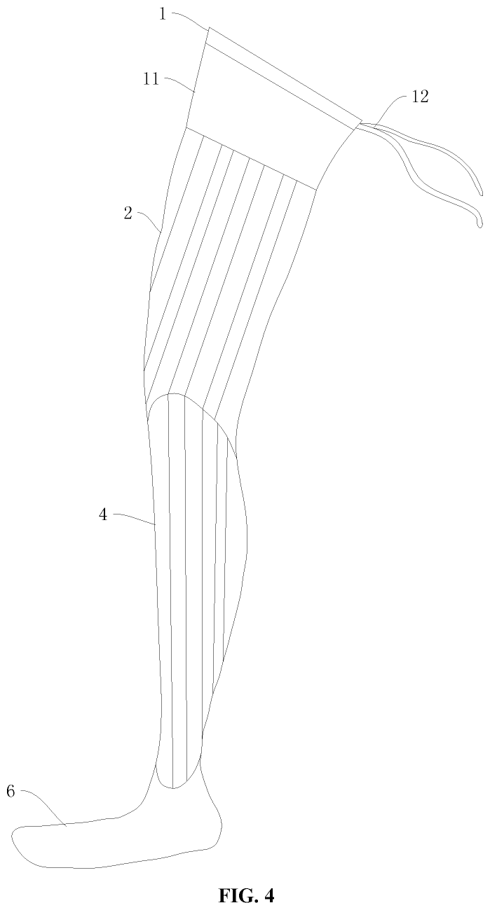

In some embodiments of the present invention, as shown in the FIG. 4, the prosthetic limb thigh 2 and the prosthetic limb shank 4 are composed of a plurality of strip-type parts, which are connected in the Velcro tape bonding form, thereby facilitating assembly and disassembly; and they can be transported separately to provide convenience for a manufacturer.

### Embodiment 4

The embodiment, based on the technical solution of the embodiment 3, provides that a mechanical prosthetic limb further includes a universal shaft, where a flexible drive part is connected with a fourth drive shaft 551 through the universal shaft.

In some embodiments of the present invention, during the running of a flexible drive part runs, since a constantly-changed angle between a prosthetic limb thigh 2 and a prosthetic limb shank 4 makes the flexible drive part adapt to the angle between the prosthetic limb thigh 2 and the prosthetic limb shank 4 by bending constantly, the universal shaft is arranged at a joint of the flexible drive part and a fourth drive part to avoid the deformation of a flexible connecting part 32 under the long-term bending condition, resulting in influence on the overall use of a prosthetic limb arising from decline in the drive effect of the flexible drive part.

### Embodiment 5

The embodiment, based on the technical solution of the embodiment 2, provides that a waterproof gasket is arranged at a rotational joint of a fourth drive shaft 551 and a prosthetic limb sole 6.

For a joint of a prosthetic limb in some embodiments of the present invention, in the embodiment, shaft-driven rotational connection at the joint of the prosthetic limb may cause a defect that in rainy and snowy days, a user using the mechanical prosthetic limb may not easily feel whether rain, which permeates through the prosthetic limb sole 6 and a prosthetic limb shank 4 along a joint seam of the prosthetic limb, is splattered to the prosthetic limb, and after permeation, due to poor ventilation, and slow drying, space in the prosthetic limb sole 6 and the prosthetic limb shank 4 may become damp, resulting in reduction in the service life of a motor 58 arising from a short circuit of the motor 58. Therefore, in the embodiment, the waterproof gasket is arranged at the rotational joint of the fourth drive shaft 551 and the prosthetic limb sole 6, thereby preventing water from being splattered to the prosthetic limb sole 6 or the prosthetic limb shank 4 in rainy and snowy days.

### Embodiment 6

With reference to FIG. 1, based on the technical solution of the embodiment 3, this embodiment provides that a fixed component 1 includes a flexible fixed shell 11 and a fastening rope 12, where the fastening rope 12 penetrates in the flexible fixed shell 11; and the fastening rope 12 is movably connected with the flexible fixed shell 11.

In some embodiments of the present invention, when a residual limb is fixed in the prior art, arranging an accommodating cavity in which plurality of fixed bosses are arranged may cause defects that the residual limb of a user is easy to deform under the long-term extrusion of the fixed bosses, and that the fixed bosses are large in a contact area, making the user uncomfortable when wearing. In order to solve the problem, in the embodiment, the flexible fixed shell 11 is used, so that the user isn't extruded by the shell after wearing; and since a mechanical prosthetic limb is driven by a motor 58, without the application of force by the user, the fastening rope 12 is used for tying up to limit in the horizontal direction, thereby improving the comfort of the user.

### Embodiment 7

The embodiment, based on the technical solution of the embodiment 6, provides that an air vent is formed in a flexible fixed shell 11.

In some embodiments of the present invention, a user needs to wear a prosthetic limb for a long time, but a residual limb fixed in the flexible fixed shell 11 for a long time causes bacteria breeding due to poor air permeability, resulting in a disease of skin for the residual limb of the user. In order to avoid the problem, in the embodiment, the air vent is formed in the flexible fixed shell 11 to ensure the hygienic security of the user's residual limb.

### Embodiment 8

The embodiment, based on the technical solution of the embodiment 2, provides that leather layers are arranged outside a prosthetic limb sole 6, a prosthetic limb shank 4, and a prosthetic limb thigh 2.

In some embodiments of the prevent invention, since a mechanical drive part with high rigidity is used inside a prosthetic limb, during the daily assembly and disassembly of the prosthetic limb, or the tumble of a user, the user is easy to hurt once being bumped. Therefore, the leather layers are arranged outside the prosthetic limb sole 6, the prosthetic limb shank 4, and the prosthetic limb thigh 2, so that the prosthetic limb sole 6, the prosthetic limb shank 4, and the prosthetic limb thigh 2 are softer; and meanwhile, a shape of the leather can also be set to be similar to a leg of a human body, so that the prosthetic limb looks attractive in appearance.

### Embodiment 9

With reference to FIG. 1, the embodiment, based on the technical solution of the embodiment 1, provides that an anti-skidding boss 61 is arranged on a contact surface between a prosthetic limb sole 6 and the ground.

In some embodiments of the prevent invention, the prosthetic limb sole 6 is arranged to achieve the support effect on a prosthetic limb; during the walking of a user, too small frictional force between the prosthetic limb sole 6 and the ground may cause the slipping of the prosthetic limb sole 6, resulting in the tumble of the user, and even, resulting in a life risk; and in case of tumble, it is troublesome for the disabled to stand up. In order to avoid the case, in the embodiment, the anti-skidding boss 61 is arranged on the contact surface between the prosthetic limb sole 6 and the ground to increase frictional force between the prosthetic limb sole 6 and the ground, thereby ensuring stability.

### Embodiment 10

With reference to FIG. 1, the embodiment, based on the technical solution of the embodiment 9, provides that a cross section of an anti-skidding boss 61 is in a triangular shape.

In some embodiments of the prevent invention, in terms of the arrangement of the anti-skidding boss 61, the cross section may be in a rectangular shape or the triangular shape, however, compared with the anti-skidding boss with the rectangular cross section, when the anti-skidding boss with the triangular cross section contacts the ground, its acute angle contacts the ground, so that the anti-skidding boss 61 with the triangular cross section is larger in frictional force, thereby improving the stability of a prosthetic limb sole 6.

In conclusion, the embodiments of the present invention provide a mechanical prosthetic limb, which includes a prosthetic limb shank 4, a fixed component 1 for connecting the prosthetic limb shank 4 with a human body, a power-driven first drive mechanism 5 that is installed on the prosthetic limb shank 4, and a prosthetic limb sole 6 that is connected to the bottom of the prosthetic limb shank 4; the fixed component 1 is rotationally connected with the prosthetic limb shank 4 through the first drive mechanism 5; and the prosthetic limb shank 4 is rotationally connected with the prosthetic limb sole 6 through the first drive mechanism 5.

In view of the direct fixing of a prosthetic limb with a residual limb in the prior art, the prosthetic limb is extremely stiff in movement when moving, bringing inconvenience to the action of a user, and needs to be driven by the user, making the disabled take a long time to make adaptation, and causing harm to the residual limb after being used for a long time. To solve the previously described problem, in the embodiment, the prosthetic limb is driven electrically by the first drive mechanism 5 installed on the prosthetic limb shank 4, and the prosthetic limb shank 4 is respectively and rotationally connected with the fixed component 1 and the prosthetic limb sole 6, so that the prosthetic limb shank 4 can be raised under the control of the first drive mechanism 5, and meanwhile, tiptoes of the prosthetic limb sole 6 incline downwards under the control of the first drive mechanism 5; and when the prosthetic limb sole 6 lays down, tiptoes touches down to the ground firstly, thereby preventing impact on the prosthetic limb arising from the direct touchdown of the center of the prosthetic limb sole 6, ensuring the residual limb isn't hurt during long-term use, and improving the safety and comfort. In addition, the power-driven drive mechanism applied in design makes the user walk conveniently and fast, with less efforts, thereby improving the practicability.

The previous descriptions are merely the preferred embodiments of the present invention, which are not intended to limit the present invention. For a person skilled in the art, there are a variety of amendments and changes in the present invention. Any amendment, equivalent replacement, improvement, etc. that are made within the spirit and principle of the present invention shall fall within the scope of protection of the present invention.

1. A mechanical prosthetic limb, characterized by comprising a prosthetic limb shank, a fixed component for connecting the prosthetic limb shank with a human body, a power-driven first drive mechanism that is installed on the prosthetic limb shank, and a prosthetic limb sole that is connected to the bottom of the prosthetic limb shank; the fixed component is rotationally connected with the prosthetic limb shank through the first drive mechanism; and the prosthetic limb shank is rotationally connected with the prosthetic limb sole through the first drive mechanism.

2. The mechanical prosthetic limb according to claim 1, characterized in that the first drive mechanism comprises a motor, a first differential gear, a first drive shaft, a first bevel gear, a second bevel gear, a second drive shaft, a third drive shaft, a third bevel gear, a fourth bevel gear, a fourth drive shaft, a fifth bevel gear, and a sixth bevel gear, wherein an output shaft of the motor is connected with the sixth bevel gear; the sixth bevel gear is meshed with the fifth bevel gear; the fifth bevel gear is connected with an input shaft of the first differential gear; one output shaft of the first differential gear is connected with the second bevel gear; the first bevel gear is meshed with the second bevel gear; the fixed component is connected with the second bevel gear through the third drive shaft; the other output shat of the first differential gear is connected with the third bevel gear through the third drive shaft; the third bevel gear is meshed with the fourth bevel gear; the fourth bevel gear is connected with the fourth drive shaft; the fourth drive shaft is connected with the prosthetic limb sole after running through the prosthetic limb shank; and the fourth drive shaft is rotationally connected with the prosthetic limb sole.

3. The mechanical prosthetic limb according to claim 2, characterized by further comprising a prosthetic limb thigh rotationally connected with the prosthetic limb shank and a second drive mechanism installed inside the prosthetic limb thigh, wherein the second drive mechanism comprises a second differential gear, a flexible drive part, and a fifth drive shaft; an input end of the second differential gear is connected with the prosthetic limb thigh; one output end of the second differential gear is connected with one end of the flexible drive part; the other end of the flexible drive part is connected with the sixth bevel gear through the fifth drive shaft; and the other output end of the second differential gear is connected with the fixed component.

4. The mechanical prosthetic limb according to claim 3, characterized by further comprising a universal shaft, wherein the flexible drive part is connected with the fourth drive shaft through the universal shaft.

5. The mechanical prosthetic limb according to claim 2, characterized in that a waterproof gasket is arranged at a rotational joint of the fourth drive shaft and the prosthetic limb sole.

6. The mechanical prosthetic limb according to claim 3, characterized in that the fixed component comprises a flexible fixed shell and a fastening rope, wherein the fastening rope penetrates in the flexible fixed shell; and the fastening rope is movably connected with the flexible fixed shell.

7. The mechanical prosthetic limb according to claim 6, characterized in that an air vent is formed in the flexible fixed shell.

8. The mechanical prosthetic limb according to claim 2, characterized in that leather layers are arranged outside the prosthetic limb sole, the prosthetic limb shank, and the prosthetic limb thigh.

9. The mechanical prosthetic limb according to claim 1, characterized in that an anti-skidding boss is arranged on a contact surface between the prosthetic limb sole and the ground.

10. The mechanical prosthetic limb according to claim 9, characterized in that a cross section of the anti-skidding boss is in a triangular shape.
